(19) United States
(12) Patent Application Publication
(10) Pub. No.: US 2024/0139450 A1
(43) Pub. Date: May 2, 2024

(54) A SUPPORT FOR AN ENDOTRACHEAL TUBE

(71) Applicant: Sheila Woodley, Englewood, NJ (US)

(72) Inventor: Sheila Woodley, Englewood, NJ (US)

(21) Appl. No.: 17/974,782

(22) Filed: Oct. 27, 2022

(51) Int. Cl. A61M 16/04

(52) U.S. Cl. CPC A61M 16/0402

(57) ABSTRACT

A support for an endotracheal tube includes a base. The base having a height when resting on a chest of the patient greater than the height of the mouth of the patient from a plane extending from their chest when lying on their back. A first member extends from the base in a direction away from the base. A second member spaced from the first member extends from the base in the direction away from the base. A gap adapted to receive the endotracheal tube therein is formed along the base between the first member and the second member.

### BACKGROUND OF THE INVENTION

The present invention is directed to a medical device for preventing necrosis, and more particularly to a support for an endotracheal tube for use in situ.

It is well known to use an endotracheal tube (“ETT”), a tube, usually constructed of polyvinylchloride (PVC) that is placed in the mouth, or between the vocal cords through the trachea, of a patient to provide oxygen and inhaled gases to the lungs, to provide ventilation to patients; particularly drug induced coma patients. Use of ETTs exploded during the Corona virus (“Covid”) epidemic as more and more patients needed help breathing, or were placed in comas.

While the performance of the ETT has been satisfactory, it can result in skin necrosis if the tube is not moved for long periods of time. Applicant has noticed an increase in necrosis, particularly of the lip, as a result of the large number of Covid patients relying on ETTs and being unable to adjust the tubes to prevent necrosis, and the inability of staff to keep up with the need to adjust the ETT on a periodic basis.

Accordingly, a system which overcome the shortcomings of the prior art is desired.

### SUMMARY OF THE INVENTION

A support for an endotracheal tube includes a base. The base having a height when resting on a chest of the patient greater than the height of the mouth from a plane extending from their chest when lying on their back. A first member extends from the base in a direction away from the base. A second member spaced from the first member extends from the base in the direction away from the base. A gap adapted to receive the endotracheal tube therein is formed along the base between the first member and the second member.

In one embodiment the support is formed of a lightweight material, soft to the touch, but substantially firm to support the endotracheal tube thereon, such as foam or foam rubber. In another embodiment, the support may be inflatable.

### BRIEF DESCRIPTION OF THE DRAWINGS

The features and advantages of the present invention will become more readily apparent from the following detailed description of the invention in which like elements are labeled similarly and in which:

FIG. 1 is a top perspective view of a support for an endotracheal tube constructed in accordance with the invention.

### DETAILED DESCRIPTION OF THE PREFERRED EMBODIMENTS

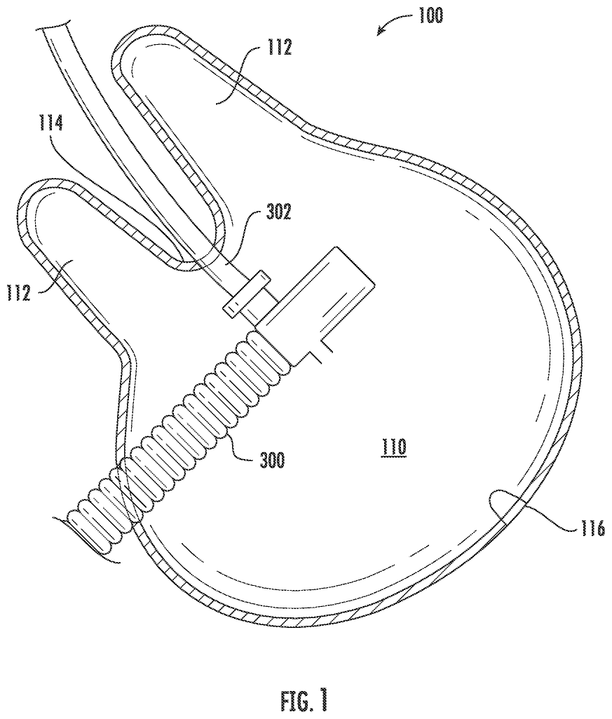

Reference is now made to FIG. 1 in which a support for an endotracheal tube, generally indicated as 100, is shown. Endotracheal tube support 100 includes a base 110. A first member 112, integrally formed with base 110, extends in a direction away from base 110. A second member 114, integrally formed with base 110, extends in a direction away from base 110. Second member 114 is spaced from first member 112 to form a gap 114 therebetween. Gap 114 is at least as wide as an endotracheal tube 302, which is usually coupled to a catheter 300, or other input to support the endotracheal tube 302 therein.

In a preferred non limiting embodiment, endotracheal tube support 100 positions the endotracheal tube 302 so as not to rest on the lip or chest of the patient. Therefore, the height of tube support 100 is at least as great as a height from a patient's mouth to a plane extending from the patient's chest when the patient is lying on their back.

Tube support 100 is formed of a lightweight, preferably low cost, material, so as not to put excessive weight on the patient during use, but sufficiently firm to support endotracheal tube 302 thereon. In a preferred non limiting embodiment, tube support 100 is formed of a foam or a foam rubber.

In a preferred non limiting embodiment, tube support 100 is formed of latex or some other elastic material and is inflatable. To that end tube support 100 includes an inflation port 116 for inflating tube support 100 for use. In this way, not only can tube support 100 be easily stored in a non inflated state, but a nurse can control the height of base 110 as a function of the amount of inflation.

It will thus be seen that the objects set forth above, among those made apparent from the preceding description, are efficiently attained and, since certain changes may be made in carrying out the above method and in the construction set forth without departing from the spirit and scope of the invention, it is intended that all matter contained in the above description and shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between.

What is claimed is:

1. A support for an endotracheal tube comprising;
a base;
a first member extends from the base in a direction away from the base;
a second member spaced from the first member extends from the base in the direction away from the base;
a gap, adapted to receive the endotracheal tube therein, is formed along the base between the first member and the second member.

2. The support of claim 1, wherein the base has a height, when resting on a chest of a patient greater than the height of the mouth of the patient from a plane extending from the chest when lying on their back.

3. The support of claim 1, wherein the support is made of one of a foam and a foam rubber.

4. The support of claim 1, wherein the support is made of an elastic material, and further comprising an inflation port.
